(19) United States
(12) Patent Application Publication
(10) Pub. No.: US 2007/0239201 A1
(43) Pub. Date: Oct. 11, 2007

(54) EMBOLUS EXTRACTOR

(75) Inventors: Mark Minh Phung, Union City, CA (US); Pete Phong Pham, Fremont, CA (US); Mehran Bashiri, San Carlos, CA (US); Ajitkumar B. Nair, Fremont, CA (US); Beth Camins, Fremont, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/764,673

(22) Filed: Jun. 18, 2007

Related U.S. Application Data
(63) Continuation of application No. 10/341,084, filed on Jan. 13, 2003, now abandoned.
Application No. 10/341,084 is a continuation-in-part of application No. 10/045,565, filed on Oct. 19, 2001, now Pat. No. 7,052,500.

(51) Int. Cl. A61M 29/00

(52) U.S. Cl. 606/200

(57) ABSTRACT

An embolus extractor including elongated shaft having a proximal end and a distal end. The embolus extractor may include first and second struts coupled to the distal end of the shaft. The struts may define a proximally disposed mouth.

This application is a continuation application of U.S. application Ser. No. 10/341/084, filed Jan. 13, 2003, which is a continuation-in-part of U.S. application Ser. No. 10/045,565, filed Oct. 19, 2001, now U.S. Pat. No. 7,052,500.

### BACKGROUND OF THE INVENTION

The present invention pertains generally to emboli collection and removal.

Blood thrombus, may form a clot in a patient vasculature. Sometimes such clots are harmlessly dissolved in the blood stream. At other times, however, such clots may lodge in a blood vessel where they can partially or completely occlude the flow of blood. If the partially or completely occluded vessel feeding blood to sensitive tissue such as, the brain, lungs or heart, for example, serious tissue damage may result.

When symptoms of an occlusion are apparent, such as an occlusion resulting in a stroke, immediate action should be taken to reduce or eliminate resultant tissue damage. One approach is to treat a patient with clot dissolving drugs. These drugs, however, do not immediately dissolve the clot and may have harmful side effects. Thus, it may be desirable to physically remove the clot from the patient.

### SUMMARY OF THE INVENTION

The present invention pertains to an improved clot or embolus extractor device and method. Various embodiments of the claimed invention are possible, examples of these embodiments will briefly be described herein and in more detail below in the detailed description of the invention. One embodiment of an embolus extractor in accordance with the invention includes two main struts coupled to the distal end of an elongated shaft. In a first collapsed position, the main struts are generally disposed parallel to the elongated shaft. In a second expanded position, the proximal end of the struts defines a generally circular mouth disposed at approximately 90° to the length of the elongated shaft. The portion of the struts extending distally of the mouth defines a generally tapered, for example, cylindrical body with a conical tip. One or more pairs of supporting struts may be attached to the main struts to more completely define a conical filter in the second expanded position. With such a configuration, an emboli mass, such as a cylindrical thrombus may be contained by the embolus extractor. One embodiment includes radiopaque markings on the proximal portions of the struts.

One embodiment of an embolus extractor in accordance with the present invention includes an elongated shaft having a proximal end and a distal end. The proximal ends and distal ends of first and second main struts are coupled to the shaft and allow rotation of the struts around the shaft. One or more pairs of supporting struts are coupled to the main struts. A sleeve may be used to slidably couple the distal ends of the struts to the shaft. A sleeve may also be used to slidably couple the proximal ends of the struts to the shaft. The struts can be disposed in a first position and a second position. In the first position, the distal ends and the proximal ends of the struts are spaced at a first distance. In the second position, the distal ends and the proximal ends of the struts are spaced at a second distance, which is less than the first distance.

In the first position, the main and supporting struts can be disposed generally parallel and adjacent to the shaft. In the second position, a proximal portion of the first and second struts can define a generally circular mouth. In the second position, the portion of the struts extending generally distally from the mouth can define a generally distally tapering body. The proximal portion of the struts forming the mouth can extend from the shaft at 45° to 90° to the length of the shaft. This angle could also be between 60° and 90° or between 80° and 90°.

The struts can include a shaped memory metal, such as NiTi alloy. Additional struts can be added to the embolus extractor to enhance the thrombus containing ability of the embolus extractor. These struts may have a smaller cross sectional diameter than the first and second struts.

In accordance with the present invention, an embolus extractor can be advanced through a patient's vasculature in a first compressed position, distally beyond a clot. The embolus extractor can then be deployed in a second expanded position, then drawn proximally to a second compressed position to capture, contain and remove the thrombus to a larger diameter vessel or from the body.

### BRIEF DESCRIPTION OF THE DRAWINGS

FIG. 1 is a prospective view of a first embodiment of an embolus extractor.

FIG. 2 is a side view of the embolus extractor of FIG. 1.

FIG. 3 is a cross sectional view of a micro catheter containing the embolus extractor of FIG. 1.

FIG. 4 is a cross sectional view of the micro catheter of FIG. 2 showing the embolus extractor partially disposed from the micro catheter.

FIG. 5 is a cross sectional view of a vessel including a clot and the embolus extractor of FIG. 1 disposed in a micro catheter positioned proximally of the clot.

FIG. 6 is a cross sectional view of the vessel of FIG. 5 showing the micro catheter and embolus extractor traversing the clot.

FIG. 7 is a cross sectional view of the vessel of FIG. 5 showing the embolus extractor deployed distally of the clot.

FIG. 8 is a cross sectional view of the vessel of FIG. 5 showing the clot captured by the embolus extractor and the extractor puller locked at the tip of the micro catheter.

FIG. 9 is a side view of an alternate embodiment of an embolus extractor.

FIG. 10 is a top view of the embolus extractor of FIG. 9.

FIG. 11 is a distal end view of the embolus extractor of FIG. 9.

FIG. 12 is a side view of an alternate embodiment of an embolus extractor.

FIG. 13 is an end view of the embolus extractor of FIG. 12.

### DETAILED DESCRIPTION

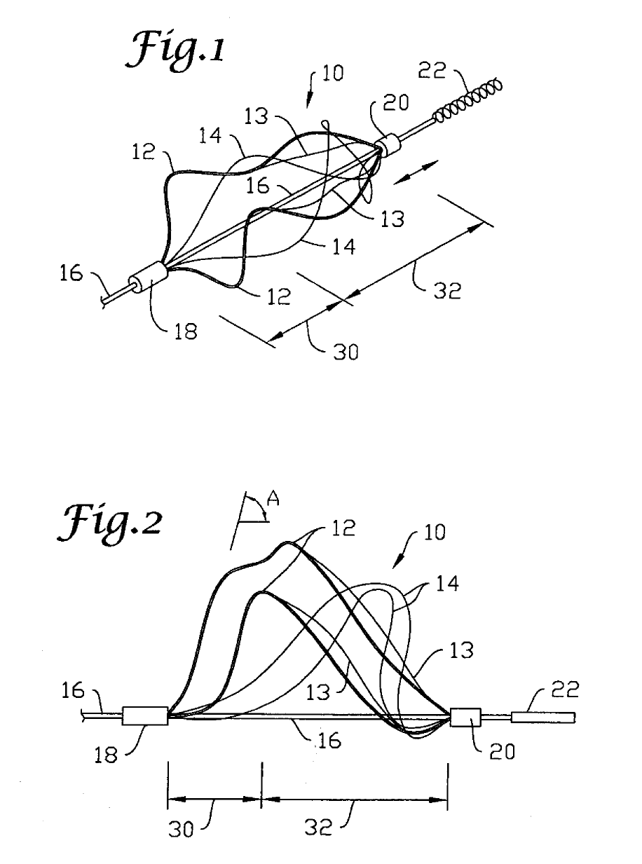

Referring now to the Figures, wherein like referenced numerals refer like elements throughout the several views, FIG. 1 is a perspective view of an embolus extractor 10. Embolus extractor 10 includes first and second primary struts 12 and first and second secondary struts 14 coupled to an elongated shaft 16, and first and second support struts 13 coupled to first and second primary struts 12. Struts 12 and 14 can be coupled to shaft 16 at their proximal ends by a sleeve 18 and at their distal ends by a sleeve 20. For example, a spring tip 22 can be disposed at the distal end of shaft 16. Spring tip 22 can be selectively shaped by a physician to guide embolus extractor 10 into micro vessels and stabilize embolus extractor 10 after deployment. Alternately, a radiopaque polymer could be used rather than a spring.

Struts 12 as shown in FIG. 1 are disposed in an expanded or delivered position. In this position, a proximal portion 30 extends generally perpendicularly to the length of shaft 16 to form a generally circular mouth 17. A distal portion 32 of struts 12 extending distally of the mouth generally tapers distally to form a distally tapered body having, for example, a generally conical distal shape. Struts 13 and 14 transverse the taper body to enhance the clot catching and holding ability of embolus extractor 10. Struts 12, 13 and 14 can be made from various materials including shaped memory metals, such as NiTi alloys. Struts 13 and 14 may have a smaller diameter or transverse cross sectional area than primary struts 12.

Elongated shaft 16 can be formed from a material similar to those used for making guide wires, such as plastic polymers, stainless steel, NiTi alloy or other suitable material. Sleeve 18 can be formed from a wire coil. Adhesive, solder or the like may be applied to fixedly connect the proximal ends of struts 12 and 14 and sleeve 18 to shaft 16 or the proximal bushing. Sleeve 20 can also be formed from a wire coil. Adhesive, solder or the like can be used to connect struts 12 and 14 to sleeve 20. If struts 12 and 14, are connected to each other, but not fixedly connected to shaft 16, sleeve 20 can slide along shaft 16. Both sleeves 18 and 20 can include a radiopaque material. Struts 12, 13 and 14 can also include radiopaque material to visualize their deployed shape.

FIG. 2 is a side view of embolus extractor 10 of FIG. 1. In FIG. 2 embolus extractor 10 is also shown in the expanded or deployed position. Proximal portion 30 of struts 12 defining the mouth is shown disposed at Angle A relative to the length of shaft 16. Angle A can be approximately 90°, between 45° and 90°, between 60° and 90°, or between 80° and 90°. It should be understood that, although Angle A is shown as the angle between the distal end of shaft 16 and proximal portion 30 of struts 12, Angle A can also be the angle between portion 30 of struts 12 and the portion of shaft 16 proximal struts 12. Since each strut 12 defining the generally circular mouth can move independently, the size of the mouth opening can vary. For example, in relatively small vessels, struts 12 can move closer together to create a smaller mouth; whereas in larger vessels, struts 12 can expand to create a larger mouth. If for example, NiTi alloy is used to form struts 12, 13 and 14, struts 12, 13 and 14 can have a preset expanded shape.

The length of shaft 16 and the size of the various elements of embolus extractor 10 can be selected with respect to the location in a patient's vasculature to be accessed. For example, if a patient's cerebral arteries are to be accessed from a femoral approach, the length of shaft 16 should be sized accordingly. The diameter of the generally circular mouth from the proximal portion 30 of struts 12 can be sized to atraumatically engage the wall of the vessel in which it is deployed. The number of primary, secondary and support struts may be increased or decreased depending on the size of the vessel and the characteristics of the clot.

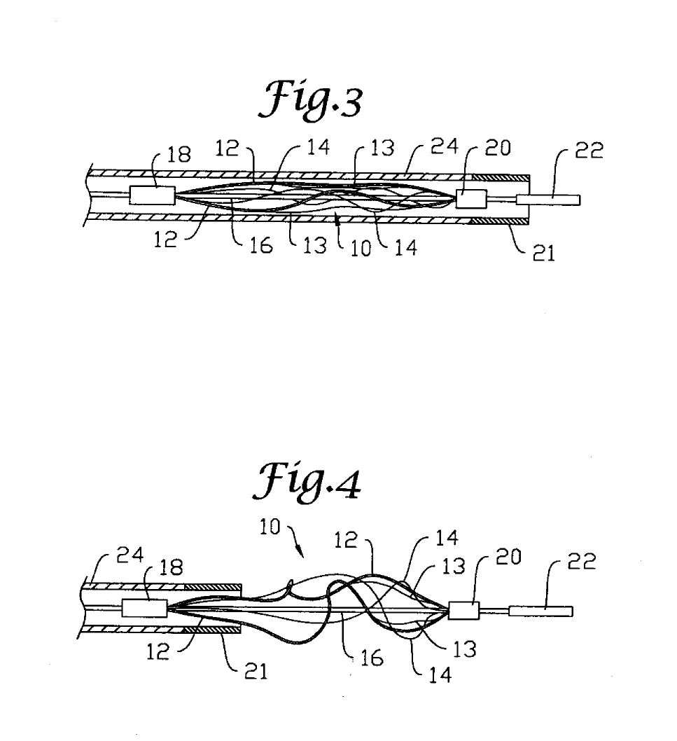

FIG. 3 is a cross sectional view of a micro catheter 24 for embolus extractor 10. Micro catheter 24 can have a radiopaque marker tip 21. Tip 21 can be made from, for example, a platinum band or a polymer loaded with a radiopaque material. As shown in FIG. 3, embolus extractor 10 is disposed in a collapsed or delivery position. In this position, sleeve 20 has slide distally along shaft 16 to allow struts 12, 13 and 14 to be compressed within micro catheter 24 and be disposed generally parallel to shaft 16. FIG. 4 is a cross sectional view of micro catheter 24 wherein embolus extractor 10 is disposed in part within micro catheter 24 and in part distally of micro catheter 24. Struts 12, 13 and 14 can be biased to self expand as micro catheter 24 is removed.

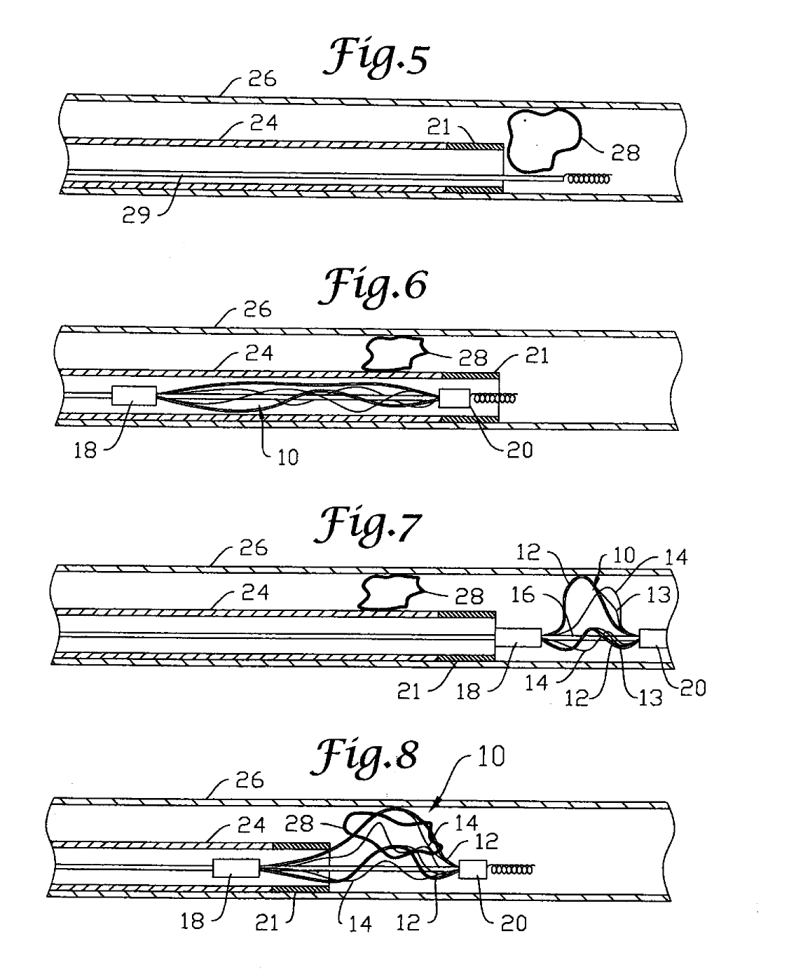

FIG. 5 is a cross sectional view of a blood vessel 26 which may be, for example, a cerebral artery. A clot 28, including thrombus is shown occluding vessel 26. A micro guidewire 29 has been advanced distally of clot 28. Micro catheter 24 will then also be advanced distally of clot 28. In some embodiments, micro guidewire 29 may be shaft 16.

As shown in FIG. 6, micro catheter 24 has been advanced distally of clot 28. Micro guidewire 29 has been removed proximally. Embolus extractor 10 has been placed in micro catheter 24 by an introducer sheath (not shown) at the proximal end of micro catheter 24.

As shown in FIG. 7, once micro catheter 24 and embolus extractor 10 are advanced at least in part distally of clot 28, embolus extractor 10 may be deployed by further advancing embolus extractor 10 relative to micro catheter 24 such that struts 12, 13 and 14 are allowed to expand. Alternately, micro catheter 24 can be retracted proximally relative to embolus extractor 10 to allow struts 12, 13 and 14 to expand.

As shown in FIG. 8, embolus extractor 10 can then be drawn proximally such that struts 14, 13 and 12 engage and capture clot 28. If struts 12 have been configured such that the proximal mouth engages the wall of vessel 26, the mouth portion can act as a separator to release clot 28 from the vessel wall. After clot 28 has been captured by embolus extractor 10, the profile of struts 12, 13 and 14 can be reduced by placing struts 12, 13 and 14, at least in part, in micro catheter. If sleeve 18 and tip 21 are radiopaque, the relative distance that embolus extractor 10 is withdrawn within micro catheter 24 can be observed by fluoroscopy. Clot 28, embolus extractor 10 and micro catheter 24 can then be removed proximally by way of, for example, a guide catheter (not shown).

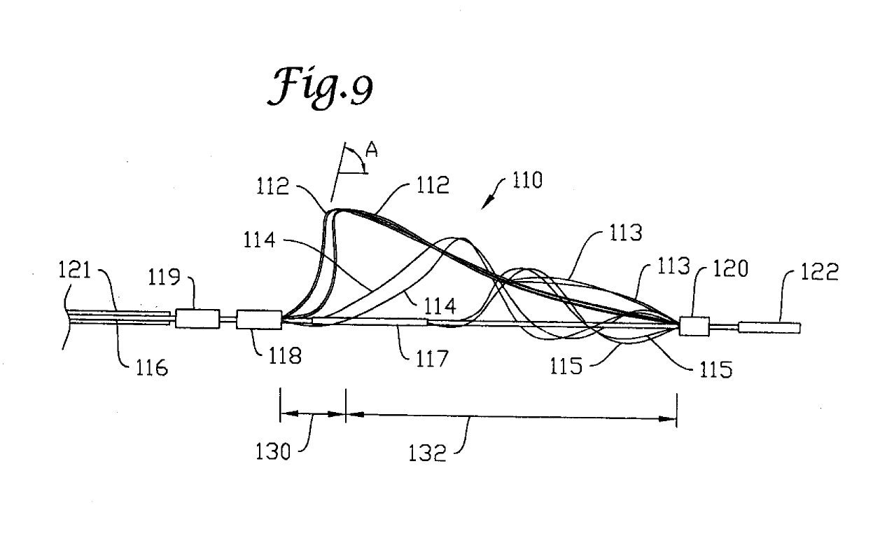

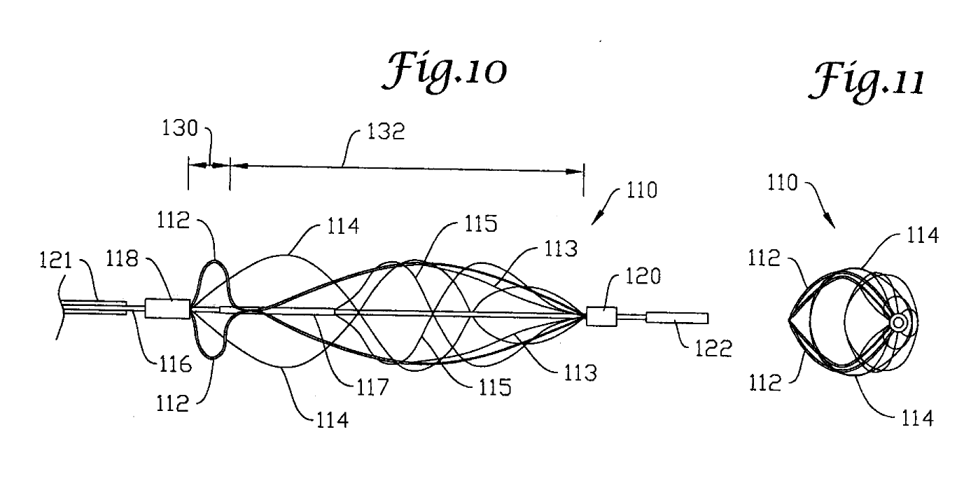

FIG. 9 is a side view of an alternate embodiment of an embolus extractor 110. Extractor 110 could also be used as an embolic protection filter. Embolus extractor 110 can be made from materials, and in an expanded position used in a manner similar to embolus extractor 10. It may include primary struts 112, support struts 113, secondary struts 114, and tertiary struts 115. Primary struts 112 and secondary struts 114 are coupled to elongated shaft 116 at their proximal ends by sleeve 118 and at their distal ends by sleeve 120. Support struts 113 are disposed on primary struts 112. In this embodiment, however, both sleeves 120 and 118 are free to slide along shaft 116. In an alternate embodiment, either sleeve 118 or 120 is fixed relative to shaft 116, or both sleeves 118 and 120 are fixed relative to shaft 116. Proximal movement, however, can be limited by a stop 119 fastened to elongated shaft 116. Tertiary struts 115 are coupled to sleeve 120 at their distal ends and to sleeve 117 at their proximal ends. Distal movement can be limited by spring tip 122. Alternately, a radiopaque polymer could be used rather than a spring. Like shaft 16, shaft 116 can be formed from a wire.

Shaft 116 can include a polymer coating 121 to improve collapse and repositioning processes of the device. Coating 121 can be polytetrafluoroethylene (PTFE) or other suitable material. Such a coating could be used on any of the shafts described herein.

As may be seen more clearly with reference to FIGS. 10 and 11, which show a top view and an end view of the embodiment, respectively, a proximal end 130 of struts 112 defines a generally circular mouth. A distal portion 132 of struts 112 and struts 113 can define a generally tapered body portion. The mouth portion of embolus extractor 110 can be disposed at an Angle A to shaft 116 as described above with respect to Angle A and embolus extractor 10.

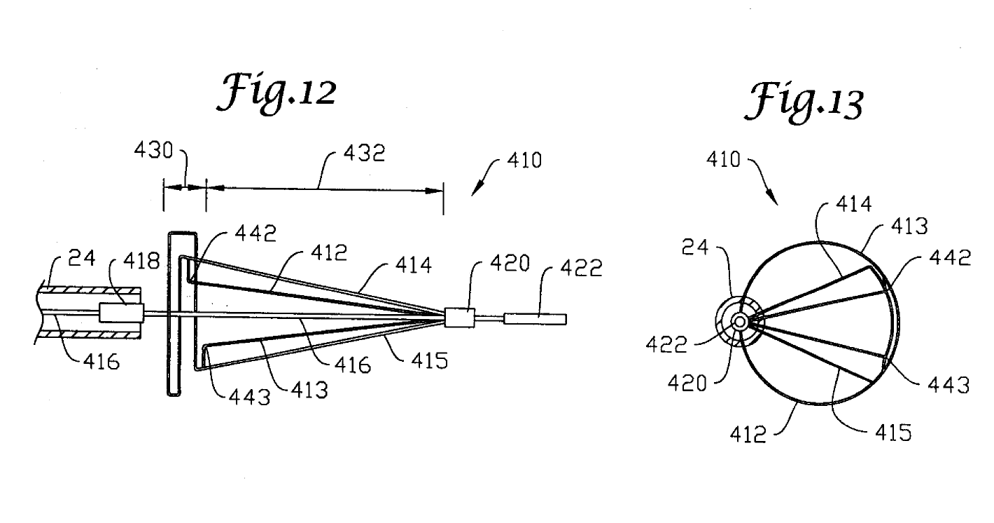

FIG. 12 is a top view of yet another alternate embodiment of an embolus extractor 410 in an expanded position. Embolus extractor 410 can be made from materials similar to, and used in a manner similar to embolus extractor 10 as described above. Embolus extractor 410 includes primary struts 412 and 413. Primary struts 412 and 413 can be coupled to an elongated shaft 416 at their proximal ends by sleeve 418 and at their distal ends by sleeve 420. Support struts 414 and 415 may be disposed on the distal portions of primary struts 412 and 413. Sleeve 418 or sleeve 420 can be slidable along shaft 416. In an alternate embodiment, either sleeve 418 or sleeve 420 is fixed relative to shaft 416, or both sleeves 418 and 420 are fixed relative to shaft 416. It may be desirable, however, if both sleeve 418 and 420 are slidable along shaft 416 to provide a stop proximal sleeve 418. A distal spring tip 422 can act as a distal stop. Alternately, a radiopaque polymer could be used rather than a spring. Proximal portion 430 of primary struts 412 and 413 can form a generally circular mouth. Distal portion 432 of primary struts 412 and 413 and struts 414 and 415 can taper distally to form a generally tapered body. A transition between proximal portion 430 and distal portion 432 can occur at bend 442 along primary strut 412 and at bend 443 along primary strut 413.

FIG. 13 is a distal end view of embolus extractor 410. The circular mouth and tapered body defined by struts 412 and 413 can be seen in FIG. 13. Additionally, it can be seen that strut 413 in part overlaps strut 412.

It should be understood that this disclosure is, in many respects, only illustrative. Changes may be made in details, particularly in matters of shape, size, and arrangement of steps without exceeding the scope of the invention. The inventor's scope is, of course, defined in the language in which the pending claims are expressed.

We claim:

1. An embolus extractor, comprising:
an elongated shaft having a proximal end and a distal end;
first and second struts, each of the first and second struts having a proximal end coupled to the shaft and a distal end coupled to the shaft;
the first and second struts having a first position and a second position, wherein in the first position, the distal ends and the proximal ends of the struts are spaced at a first distance, and in the second position the distal ends and the proximal ends of the struts are spaced at a second distance, the second distance being less than the first distance; and
third and fourth struts, each of the third and fourth struts coupled to one of the first and second struts at a location intermediate the proximal end and distal end of the first and second struts.

2. An embolus extractor in accordance with claim 1, further comprising a sleeve slidably coupling the distal ends of the first and second struts to the shaft.

3. An embolus extractor in accordance with claim 1, further comprising a sleeve slidably coupling the proximal ends of the first and second struts to the shaft.

4. An embolus extractor in accordance with claim 1, wherein in the first position, the struts are disposed generally parallel to and adjacent the shaft.

5. An embolus extractor in accordance with claim 1, wherein in the second position, a proximal portion of the first and second struts define a generally circular mouth.

6. An embolus extractor in accordance with claim 5, wherein the first and second struts extend generally distally from the mouth to define a generally distally tapering body.

7. An embolus extractor in accordance with claim 5, wherein the proximal portion of the first and second struts forming the mouth extend from the shaft at between 45° to 90° the length of the shaft.

8. An embolus extractor in accordance with claim 7, wherein the proximal portions of the first and second struts forming the mouth extend from the shaft at between 60° to 90° to the length of the shaft.

9. An embolus extractor in accordance with claim 8, wherein the proximal portions of the first and second struts forming the mouth extend from the shaft at between 80° to 90° to the length of the shaft.

10. An embolus extractor in accordance with claim 1, wherein the struts include a shape memory metal.

11. An embolus extractor in accordance with claim 10, wherein the shape memory metal includes a NiTi alloy.

12. An embolus extractor in accordance with claim 1, wherein the third and fourth struts each have a middle region spaced apart from the first and second struts when the first and second struts are in the second position.

13. The embolus extractor in accordance with claim 1, wherein the first and second struts form at least a portion of a generally circular mouth.

14. The embolus extractor in accordance with claim 13, wherein the first and second struts can move independently of each other.

15. The embolus extractor in accordance with claim 1, wherein the first and second struts can rotate about the elongated shaft.

16. The embolus extractor in accordance with claim 1, wherein the first and second struts can translate at least in part along the elongated shaft.

17. The embolus extractor in accordance with claim 1, wherein at least one strut includes a radiopaque material.

18. An embolus extractor, comprising:
an elongated shaft having a proximal end and a distal end;
a first strut having a proximal end and a distal end, the proximal end of the strut being coupled to the shaft;
the first strut having a first position and a second position, wherein in the first position, the distal end and the proximal end of the first strut are spaced at a first distance, and in the second position, the distal end and the proximal end of the first strut are spaced at a second distance being less than the first distance;
a second strut having a proximal end and a distal end, the proximal end of the second strut being coupled to the first strut at a location intermediate the proximal end and distal end of the first strut.

19. An embolus extractor in accordance with claim 18 wherein in the first position, the struts are disposed generally parallel to the shaft.

20. An embolus extractor in accordance with claim 18, wherein in the second position, a proximal portion of the first strut defines a portion of a generally circular mouth.

21. An embolus extractor in accordance with claim 20, wherein the first strut extends generally distally from the mouth to define a generally distally tapering body.

22. An embolus extractor in accordance with claim 20, wherein the proximal portion of the first strut forming the mouth, extends from the shaft at between 45° to 90° to the length of the shaft.

23. An embolus extractor in accordance with claim 22, wherein the proximal portion of the first strut forming the mouth, extends from the shaft at between 60° to 90° to the length of the shaft.

24. An embolus extractor in accordance with claim 23, wherein the proximal portion of the first strut forming the mouth, extends from the shaft at between 80° to 90° to the length of the shaft.

25. An embolus extractor in accordance with claim 18, wherein at least one of the first and second struts includes a shape memory metal.

26. An embolus extractor in accordance with claim 25, wherein the shape memory metal includes a NiTi alloy.

27. An embolus extractor in accordance with claim 18, further comprises a third strut coupled to the shaft, the second strut having a transverse cross sectional area; wherein the first strut has a transverse cross sectional area greater than the cross sectional area of the second strut.
